(19) United States
(12) Patent Application Publication
(10) Pub. No.: US 2026/0174794 A1
(43) Pub. Date: Jun. 25, 2026

(54) SELENIUM-CONTAINING HYPERBRANCHED POLYMER (HBP), AND PREPARATION METHOD AND APPLICATION THEREOF

(71) Applicant: HANGZHOU NORMAL UNIVERSITY, Hangzhou City (CN)

(72) Inventors: Junqiu LIU, Hangzhou City (CN); Cong LI, Hangzhou City (CN); Yaqi WU, Hangzhou City (CN); Sheng BAO, Hangzhou City (CN); Jiayun XU, Hangzhou City (CN); Yongfeng ZHOU, Hangzhou City (CN); Tengfei YAN, Hangzhou City (CN); Tingting WANG, Hangzhou City (CN)

(73) Assignee: HANGZHOU NORMAL UNIVERSITY, Hangzhou City (CN)

(21) Appl. No.: 19/427,768

(22) Filed: Dec. 19, 2025

(30) Foreign Application Priority Data
Dec. 25, 2024 (CN) 202411927323.5

(51) Int. Cl. A61K 31/80; C08G 65/46

(52) U.S. Cl. CPC A61K 31/80; C08G 65/46

(57) ABSTRACT

A selenium-containing hyperbranched polymer (HBP), and a preparation method and application thereof are provided, belonging to the technical field of intelligent biomimetic materials preparation. The preparation method includes the following steps: preparing a compound M1; preparing an HBP; dissolving the compound M1 in anhydrous dichloromethane (DCM), adding anhydrous N,N-dimethylformamide (DMF) and oxalyl chloride, stirring an obtained mixture, and removing the anhydrous DCM and excess oxalyl chloride under reduced pressure to obtain a reactant A; under nitrogen protection, suspending the HBP and triethylamine (TEA) in anhydrous tetrahydrofuran (THF), and stirring to obtain a reactant B; and dissolving the reactant A in anhydrous THF, dropwise adding an obtained solution to the reactant B, heating an obtained mixture to room temperature, stirring, removing the anhydrous THF, redissolving an obtained product in anhydrous THF, and conducting dialysis to obtain the selenium-containing HBP.

### CROSS REFERENCE TO RELATED APPLICATION

This patent application claims the benefit and priority of Chinese Patent Application No. 2024119273235, filed with the China National Intellectual Property Administration on Dec. 25, 2024, the disclosure of which is incorporated by reference herein in its entirety as part of the present application.

### TECHNICAL FIELD

The present disclosure belongs to the technical field of intelligent biomimetic materials preparation, and specifically relates to a selenium-containing hyperbranched polymer (HBP), and a preparation method and application thereof.

### BACKGROUND

Natural channel proteins, such as acid-sensing ion channels (ASIC), adenosine triphosphate (ATP)-sensitive potassium channels (KATP), and the human voltage-gated proton channel (hHv1), form channels in the cell membrane that control the transmembrane transport of species like ions and water molecules. These channel proteins are crucial for maintaining the normal physiological functions of organisms. Pathological disorders of these channel proteins can generally lead to a series of diseases, including cardiomyopathy and cystic fibrosis. The hHv1 channel is particularly important because it effectively regulates intracellular proton efflux, influences the production of reactive oxygen species (ROS), and thereby modulates the normal operation of other physiological activities. The dysfunction of this channel is associated with various diseases, including chronic pain.

Inspired by this, biomimetic chemists have been committed to developing artificial transmembrane transport systems to mimic the high transport efficiency, remarkable ion selectivity, and gating characteristics of natural channel proteins. However, developing artificial systems that simultaneously satisfy these characteristics is highly challenging, especially concerning the selectivity and transport rate of artificial proton channels. Hyperbranched polymers (HBPs), as three-dimensional macromolecules with internal cavities and a large number of functional groups, have garnered significant interest and found applications in fields such as drug delivery, nanomaterials, and molecular sensing. The three-dimensional structure and abundant functional groups of HBPs make them promising for mimicking the selective proton transport of natural proton channels. Furthermore, the introduction of selenium may influence ion transport by modulating the ability of HBPs to embed into phospholipid membranes, showing potential for integrating various characteristics of natural channel proteins. Therefore, synthetic systems based on selenium-containing HBPs might enable efficient and selective proton transport across lipid bilayers and cell membranes, while also exhibiting controllable behavior. This offers a promising research direction for developing novel artificial proton channels.

### SUMMARY

The present disclosure aims to provide a selenium-containing HBP, and a preparation method and application thereof. This selenium-containing HBP exhibits excellent anticancer activity by rapidly changing intracellular pH and inducing cancer cell apoptosis, and shows significant toxicity against two cancer cell lines: melanoma B16F10 and malignant glioma U87MG.

To solve the above technical problems, the present disclosure adopts the following technical solutions:

The present disclosure provides a preparation method of a selenium-containing HBP, including the following steps:
S1, preparing a compound M1;
S2, preparing an HBP;
S3, dissolving the compound M1 obtained in the step S1 in anhydrous dichloromethane (DCM), adding anhydrous N,N-dimethylformamide (DMF) and oxalyl chloride, heating and stirring an obtained mixture overnight, and removing the anhydrous DCM, the anhydrous DMF and excess oxalyl chloride under reduced pressure to obtain a reactant A;
S4, under nitrogen protection, suspending the HBP obtained in the step S2 and triethylamine (TEA) in anhydrous tetrahydrofuran (THF), and stirring at 0° C. for 5 min to obtain a reactant B; and
S5, dissolving the reactant A obtained in the step S3 in anhydrous THF, stirring uniformly, dropwise adding an obtained solution to the reactant B obtained in the step S4, heating an obtained mixture to room temperature, stirring overnight, removing the anhydrous THF using a rotary evaporator, redissolving an obtained product in anhydrous THF, and conducting dialysis for 24 h in a THF-water mixture system with a volume ratio of 1:1 to obtain the selenium-containing HBP.

Preferably, a preparation process of the compound M1 in the step S1 includes the following steps:
A1, dissolving dimethyl diselenide in anhydrous ethanol, stirring uniformly, slowly adding sodium borohydride at 0° C., and stirring at 0° C. for 30 min to obtain a mixture A;
A2, dissolving ethyl chloroacetate in anhydrous ethanol, stirring uniformly, adding an obtained solution to the mixture A obtained in the step A1, and stirring at 0° C. for 1 h to obtain a mixture B;
A3, adding distilled water and anhydrous ethanol sequentially to the mixture B obtained in the step A2, conducting extraction to separate an organic layer, washing the organic layer with distilled water and brine sequentially, retaining an obtained organic phase, and removing the anhydrous ethanol under reduced pressure to obtain a mixture C; and
A4, dissolving the mixture C obtained in the step A3 in ethanol with a mass fraction of 99%, adding an aqueous KOH solution, stirring overnight at room temperature, adding distilled water and diethyl ether sequentially, acidifying with concentrated hydrochloric acid to a pH value of 2, conducting extraction with diethyl ether, washing an obtained organic layer with distilled water, drying the organic layer over anhydrous Na₂SO₄, and removing the ethanol with a mass fraction of 99% and the diethyl ether using a rotary evaporator to obtain the compound M1.

Preferably, a preparation process of the HBP in the step S2 includes the following steps:
B1, under nitrogen protection, adding anhydrous DCM and boron trifluoride diethyl etherate to a round-bottom flask to obtain a mixture D; and
B2, adding 3-ethyl-3-(hydroxymethyl)oxetane to the mixture D obtained in the step B1 via a dropping funnel within 5 min, conducting a reaction at −20° C. to 30° C. for 48 h to 54 h, quenching the reaction with ethanol, adding an obtained reaction product to ultrapure water, filtering, and vacuum drying at 80° C. for 180 min to obtain the HBP.

Preferably, in the step S3, the compound M1 obtained in the step S1 is dissolved in the anhydrous DCM, the anhydrous DMF and the oxalyl chloride are added, and the mixture is heated and stirred under reflux at 60° C. overnight under nitrogen protection.

Preferably, the anhydrous THF and distilled water are at a volume ratio of 1:1 in the THF-water mixture system in the step S5.

Preferably, during the dialysis in the step S5, the THF-water mixture system is replaced every 3 h, and a dialysis membrane used has a molecular weight cut-off of 1,000 Da.

The present disclosure further provides a selenium-containing HBP prepared by the preparation method.

The present disclosure further provides application of a selenium-containing HBP prepared by the preparation method or the selenium-containing HBP in manufacture of a drug for treating and/or preventing a tumor.

The present disclosure further provides a pharmaceutical composition, including: a selenium-containing HBP prepared by the preparation method and the selenium-containing HBP.

Preferably, the pharmaceutical composition includes the selenium-containing HBP as an active ingredient and a pharmaceutically acceptable carrier.

A pharmaceutical formulation includes a therapeutically effective amount of the selenium-containing HBP, and a pharmaceutically acceptable excipient.

The pharmaceutical formulation includes the following dosage forms: oral formulations (such as tablets, capsules, solutions, or suspensions); injectable preparations (such as injectable solutions or suspensions, or injectable dry powder which can be used immediately after adding water for injection); topical preparations (such as ointments or solutions).

Carriers for application in the pharmaceutical compositions of the present disclosure are common carriers available in the field of pharmaceutics, including: binders, lubricants, disintegrants, solubilizing agents, diluents, stabilizers, suspending agents, colorants, and flavoring agents for oral formulations; preservatives, solubilizing agents, and stabilizers for injectable preparations; substrates, diluents, lubricants, and preservatives for topical preparations. The pharmaceutical formulations can be administered orally or parenterally (e.g., intravenously, subcutaneously, intraperitoneally, or topically). If certain drugs are unstable under gastric conditions, they can be formulated as enteric-coated tablets.

Compared with the prior art, the present disclosure has the following advantages and technical effects:

(1) The present disclosure discloses an HBP capable of rapidly and highly selectively transporting protons at a rate comparable to natural gramicidin. Meanwhile, the HBP possesses extremely high ion selectivity, and effectively distinguishes protons from other ions such as potassium ions, sodium ions, and chloride ions. By introducing selenide onto a surface of the HBP, the obtained selenium-containing HBP not only maintains efficient and selective proton transport capability but also acquires redox-regulated characteristics, enabling in situ redox switching under the alternating action of glutathione (GSH) and hydrogen peroxide (H₂O₂) to control proton transport.

(2) This selenium-containing HBP exhibits excellent anticancer activity by rapidly changing intracellular pH and inducing cancer cell apoptosis. This selenium-containing HBP shows significant toxicity against two cancer cell lines: melanoma B16F10 and malignant glioma U87MG. The technical effects of the present disclosure are reflected not only in its exceptional proton transport efficiency and selectivity but also in its unique redox regulation mechanism and remarkable anticancer efficacy. As a result, the present disclosure provides an important scientific basis and application prospects for the development of novel intelligent biomimetic material drugs.

The technical solutions of the present disclosure will be further described in detail below with reference to drawings and embodiments.

### BRIEF DESCRIPTION OF THE DRAWINGS

FIG. 1 shows the chemical structures, synthesis routes, molecular weights (Mn), and degrees of branching (DB) of the HBPs H1, H2, and H3 provided in Examples 1 to 3;

FIG. 2 shows the hydrogen nuclear magnetic resonance (¹H NMR) spectrum of the HBP H1 provided in Example 1;

FIG. 3 shows the carbon nuclear magnetic resonance (¹³C NMR) spectrum of the HBP H1 provided in Example 1;

FIG. 4 shows the gel permeation chromatography (GPC) chromatogram of the HBP H1 provided in Example 1;

FIG. 5 shows the ¹H NMR spectrum of the HBP H2 provided in Example 2;

FIG. 6 shows the ¹³C NMR spectrum of the HBP H2 provided in Example 2;

FIG. 7 shows the GPC chromatogram of the HBP H2 provided in Example 2;

FIG. 8 shows the ¹H NMR spectrum of the HBP H3 provided in Example 3;

FIG. 9 shows the ¹³C NMR spectrum of the HBP H3 provided in Example 3;

FIG. 10 shows the GPC chromatogram of the HBP H3 provided in Example 3;

FIGS. 11A-11F show the vesicle activity test results; where FIG. 11A shows a schematic diagram of the pyranine (HPTS) probe vesicle experiment; FIG. 11B shows a comparison of proton transport activity among H1 to H3 provided in Examples 1 to 3 and gramicidin A (gA) at the same concentration (3.18 μM); FIG. 11C shows the half maximal effective concentration (EC₅₀) values for H3 provided in Example 3 and gA; FIG. 11D shows the EC₅₀ values for H1 and H2 provided in Example 1 and Example 2;

FIGS. 12A-12F show the lipid bilayer proton transport rate test results for H3 provided in Example 3; where FIG. 12A shows a schematic diagram of the planar lipid bilayer workstation; FIG. 12B shows the channel current signals of H3 under different voltages when both the cis and trans chambers contain 0.25 M aqueous hydrochloric acid solution; FIG. 12C shows the current-voltage (I-V) curve used to determine the proton conductivity (γ_{H}_{⁺}) of H3; FIG. 12D shows H3 channels remaining continuously open for over 70 s, indicating transport stability results; FIG. 12E shows the channel current signals of gA under different voltages when both the cis and trans chambers contain 0.25 M aqueous hydrochloric acid solution; FIG. 12F shows the current-voltage (I-V) curve used to determine the proton conductivity (γ_{H}_{⁺}) of gA;

FIGS. 13A-13F show the lipid bilayer H⁺/Na⁺, H⁺/K⁺, and H⁺/Cl⁻ selectivity test results for H3 provided in Example 3; where FIG. 13A shows the channel current signals of H3 under different voltages when the cis chamber contains 0.25 M aqueous hydrochloric acid solution and the trans chamber contains 0.25 M aqueous potassium chloride solution; FIG. 13B shows the current-voltage (I-V) curve used to determine the H⁺/K⁺ selectivity of H3; FIG. 13C shows the channel current signals of H3 under different voltages when the cis chamber contains 0.25 M aqueous hydrochloric acid solution and the trans chamber contains 0.25 M aqueous sodium chloride solution; FIG. 13D shows the current-voltage (I-V) curve used to determine the H⁺/Na⁺ selectivity of H3; FIG. 13E shows the channel current signals of gA under different voltages when both the cis and trans chambers contain 0.25 M aqueous hydrochloric acid solution; FIG. 13F shows the current-voltage (I-V) curve used to determine the proton conductivity (γ_{H}_{⁺}) of gA;

FIGS. 14A-14E show the chemical structures of H1 to H3 and H3-Se provided in Examples 1 to 3 and Example 4, respectively, and a schematic diagram of proton transport inducing cancer cell apoptosis; where FIG. 14A shows the chemical structures, synthesis routes, molecular weights (Mn), and degrees of branching (DB) of H1, H2, and H3 provided in Examples 1 to 3; FIG. 14B shows the chemical structure and redox-regulated structural change of H3-Se provided in Example 4; FIG. 14C shows H3 transporting protons with high selectivity via multiple pathways; FIG. 14D shows a schematic diagram of redox-regulated proton transport for H3-Se provided in Example 4; FIG. 14E shows the process of H3 provided in Example 3 inducing cancer cell apoptosis;

FIG. 15 shows the synthesis routes of M1 and H3-Se in Example 4;

FIG. 16 shows the ¹H NMR spectrum of M1 provided in Example 4;

FIG. 17 shows the ¹³C NMR spectrum of M1 provided in Example 4;

FIG. 18 shows the selenium nuclear magnetic resonance (⁷⁷Se NMR) spectrum of M1 provided in Example 4;

FIG. 19 shows the mass spectrum of M1 provided in Example 4;

FIG. 20 shows the ¹H NMR spectrum of H3-Se provided in Example 4;

FIG. 21 shows the ⁷⁷Se NMR spectrum of H3-Se provided in Example 4;

FIGS. 22A-22F show the Se and ¹³C NMR spectra of redox-regulated M1, the EC₅₀ value of H3-Se, and the diagram of redox-regulated proton transport; where FIG. 22A shows the ⁷⁷Se NMR spectrum of M1; FIG. 22B shows the ¹³C NMR spectrum of M1; FIG. 22C shows the EC₅₀ value of H3-Se; FIGS. 22D-22E show the change in relative fluorescence intensity of the HPTS probe after adding different amounts of H₂O₂ to H3-Se (3.5 μM) followed by adding different amounts of GSH; FIG. 22F shows the change in transport activity of H3-Se (3.5 μM) upon cyclic addition of H₂O₂ and GSH; and

FIGS. 23A-23B show the cytotoxicity and apoptosis induction by H3; where FIG. 23A shows the dose-dependent cell viability curves of B16F10 and U87MG cancer cells after 24-h culture with H3; FIG. 23B shows the flow cytometry analysis results of U87MG cells cultured with H3 or DMSO for 24 h, followed by staining with Annexin V and propidium iodide (PI).

### DETAILED DESCRIPTION OF THE EMBODIMENTS

The technical solutions of the present disclosure will be further described below with reference to the drawings and examples.

Unless otherwise defined, the technical or scientific terms used herein should have the usual meanings understood by a person of ordinary skill in the field to which the present disclosure belongs.

The test materials used in the examples of the present disclosure are conventional test materials in the art, and are commercially available.

Example 1 This example provided an HBP H1. A preparation method included the following steps:

B1. Under nitrogen protection, 80 mL of anhydrous DCM and 6.4 mL (0.05 mol) of boron trifluoride diethyl etherate were added to a round-bottom flask to obtain a mixture D.

B2. 11.6 mL (0.1 mol) of 3-ethyl-3-(hydroxymethyl)oxetane was added to the mixture D obtained in the step B1 via a dropping funnel within 5 min. The reaction was conducted at −20° C. for 54 h, quenched with ethanol, a resulting reaction product was added to ultrapure water, filtered, and vacuum-dried at 80° C. for 180 min to obtain the HBP H1 with a yield of 83%.

Example 2 This example provided an HBP H2. A preparation method was the same as that in Example 1, except that in the step B2, the reaction was conducted at −20° C. for 48 h. The HBP H2 was obtained with a yield of 85%.

Example 3 This example provided an HBP H3. A preparation method was the same as that in Example 1, except that in the step B2, the reaction was conducted at 30° C. for 48 h. The HBP H3 was obtained with a yield of 91%.

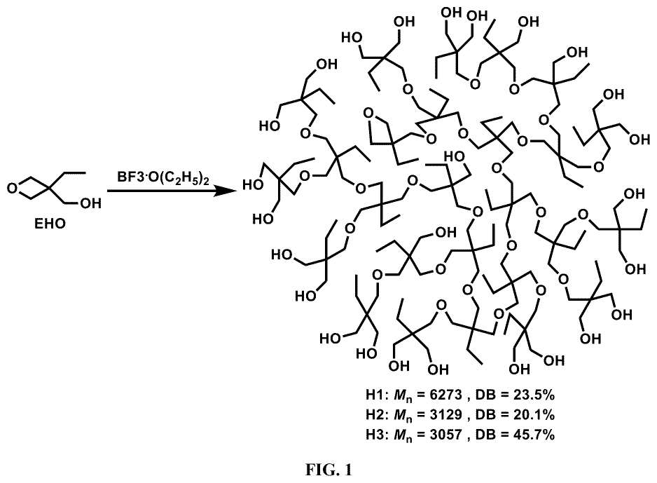

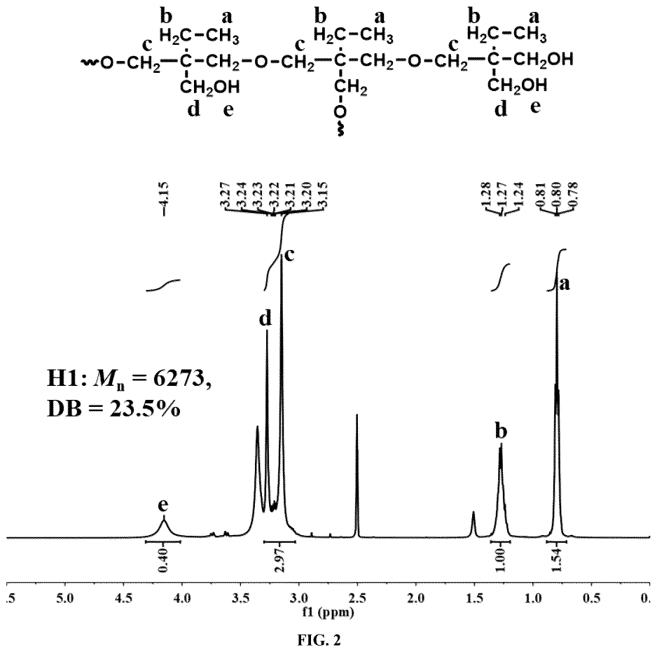

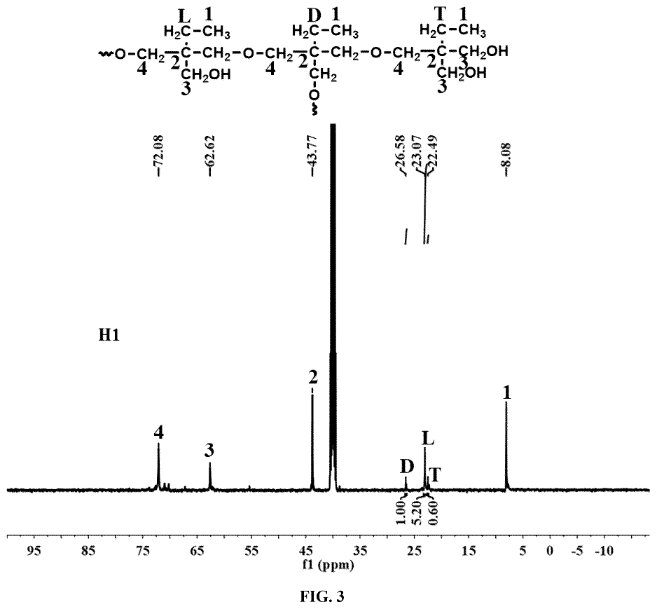

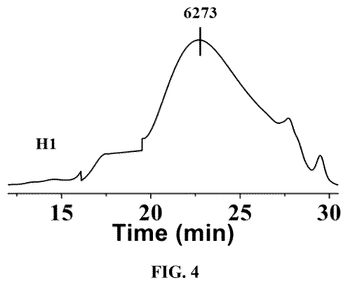

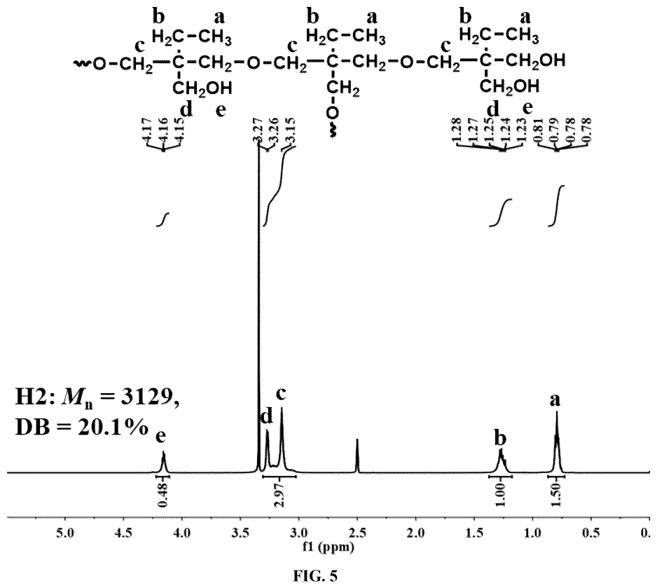

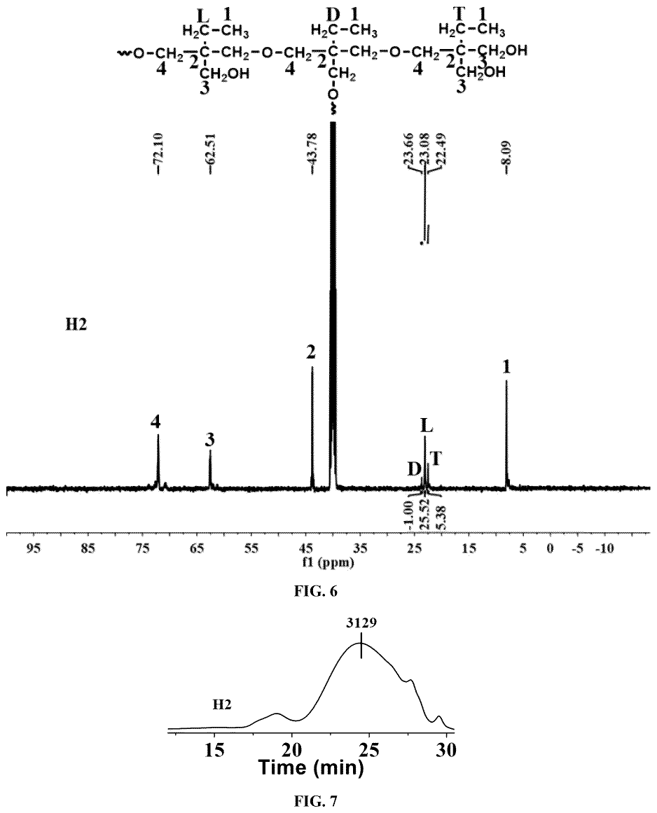

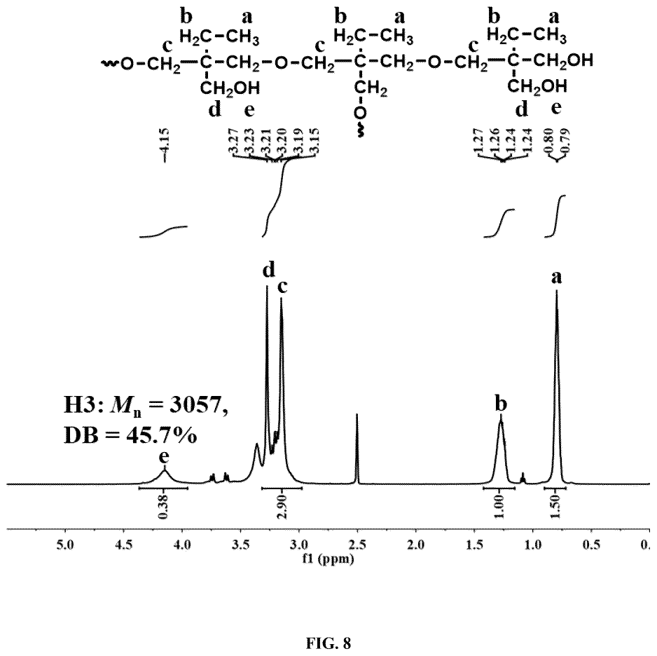

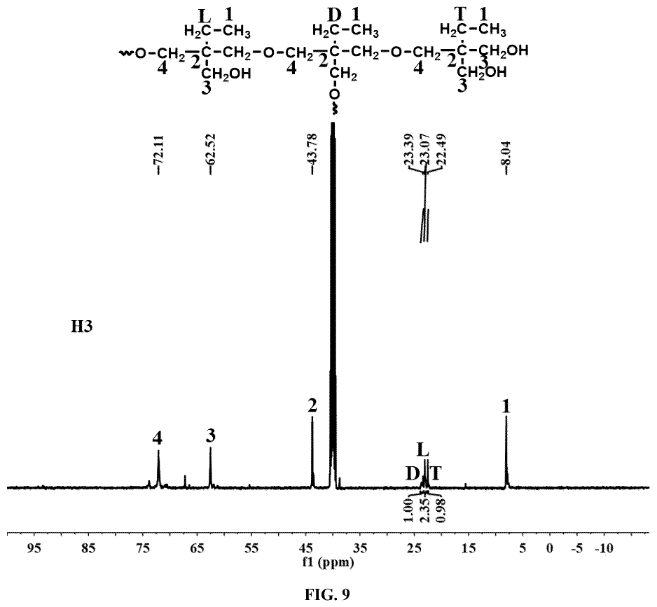

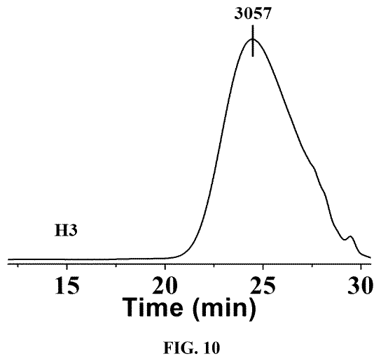

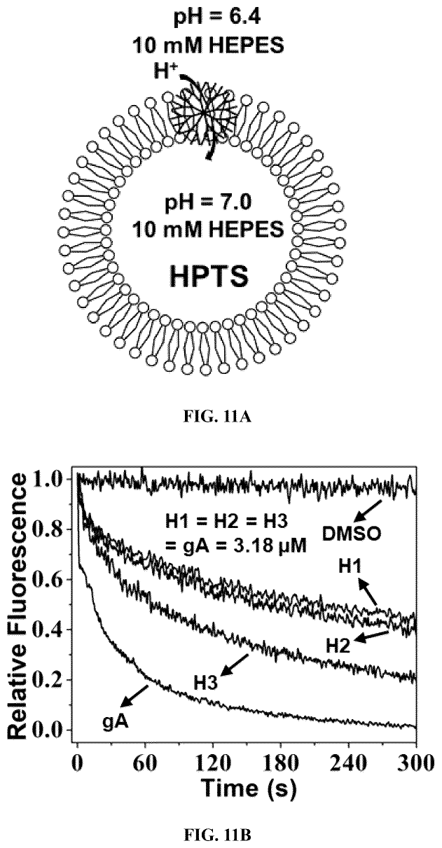

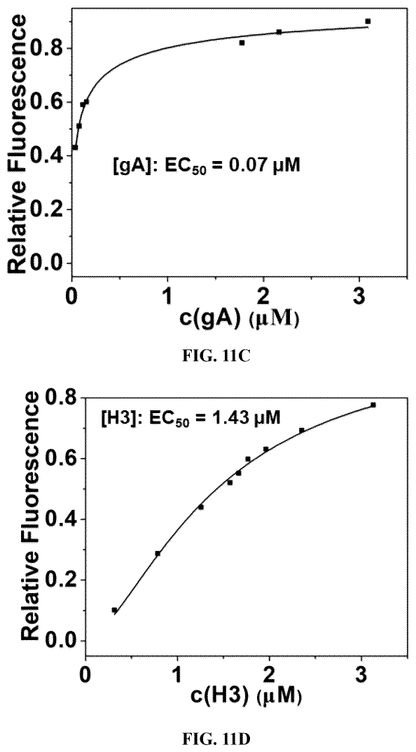

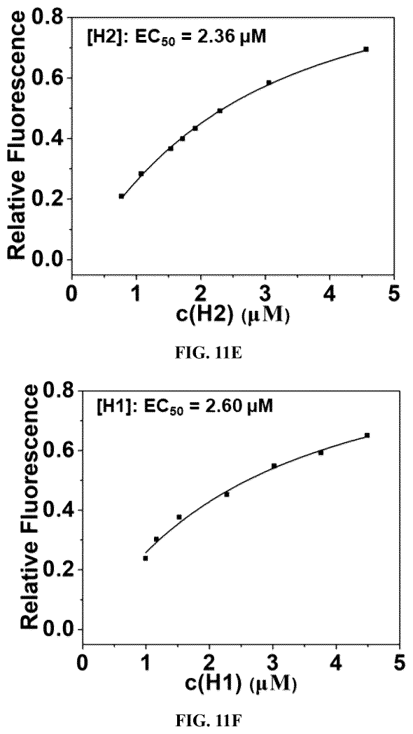

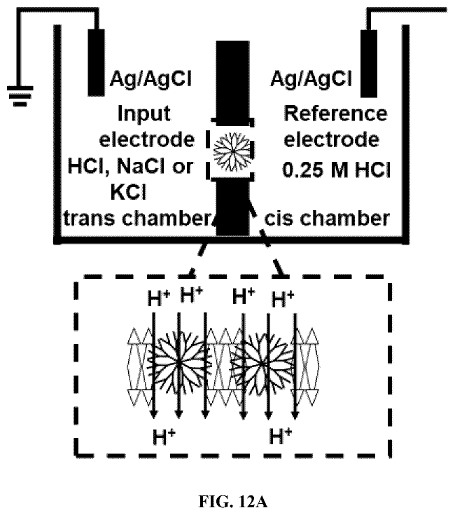

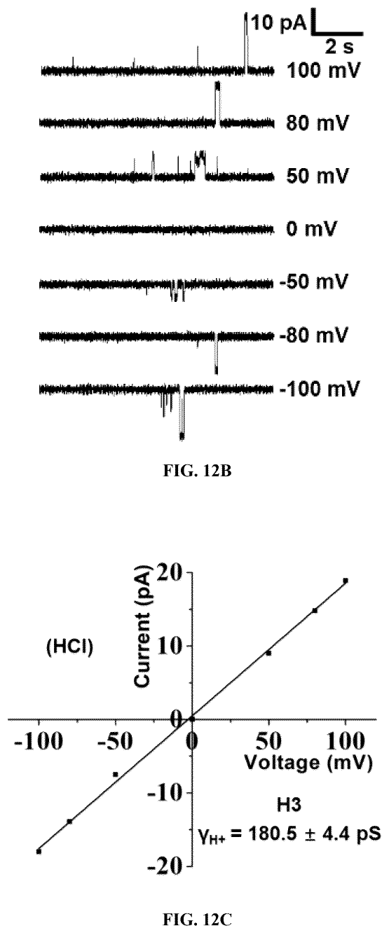

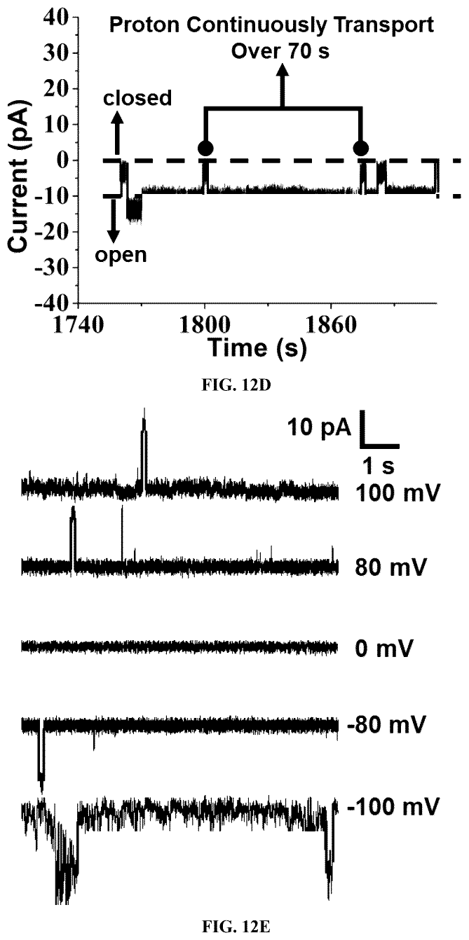

1. The HBPs H1, H2, and H3 obtained in Examples 1 to 3 were characterized by NMR and GPC. The results are shown in FIG. 1 to FIG. 10.

From FIG. 1 to FIG. 10, it was determined that the molecular weights (Mn) of H1, H2, and H3 were 6,273, 3,129, and 3,057, respectively, and the corresponding degrees of branching (DB) were 23.5%, 20.1%, and 45.7%, respectively.

H1: ¹H NMR (500 MHz, DMSO-d6) δ 4.15 (s, 1H), 3.30-3.03 (m, 3H), 1.26 (t, J=11.8 Hz, 1H), 0.80 (t, J=7.4 Hz, 2H). ¹³C NMR (500 MHz, DMSO-d6) δ 72.08, 62.62, 43.77, 26.58, 23.07, 22.49, 8.08. GPC (THF): Mn=6273, PDI=1.80. DB=23.5%.

H2: ¹H NMR (500 MHz, DMSO-d6) δ 4.22-4.11 (m, 1H), 3.31-3.02 (m, 3H), 1.37-1.18 (m, 1H), 0.79 (dd, J=9.6, 5.2 Hz, 2H). ¹³C NMR (500 MHz, DMSO-d6) δ 72.10, 62.51, 43.78, 23.66, 23.08, 22.49, 8.09. GPC (THF): Mn=3129, PDI=1.79. DB=20.1%.

H3: ¹H NMR (500 MHz, DMSO-d6) δ 4.15 (s, 1H), 3.32-2.98 (m, 3H), 1.25 (dd, J=12.2, 4.6 Hz, 1H), 0.79 (d, J=4.0 Hz, 2H). ¹³C NMR (500 MHz, DMSO-d6) δ 72.11, 62.52, 43.78, 23.39, 23.07, 22.49, 8.04. GPC (THF): Mn=3057, PDI=1.60. DB=45.7%.

2. Vesicle Activity Test: To evaluate the ion transport activity of H1 to H3 provided in Examples 1 to 3, experiments were conducted using large unilamellar vesicles (LUVs) containing the pH-sensitive 8-hydroxypyrene-1,3,6-trisulfonic acid (pyranine, HPTS) probe. The LUVs were prepared as follows: 11 mg of egg yolk L-α-phosphatidylcholine (EYPC) purchased from Sigma was dissolved in 1 mL of chloroform, and the solvent was then evaporated under vacuum using an oil pump for 3 h, resulting in a visible white film. Subsequently, 1 mL of 4-(2-hydroxyethyl)-1-piperazine-ethanesulfonic acid (HEPES) buffer solution (10 mM HEPES, pH=7.0) and the HPTS probe (1 mM) were added, and hydration was conducted in a 37° C. water bath for 3 h. This solution was then subjected to repeated freeze-thaw cycles using liquid nitrogen and a constant-temperature water bath (37° C.), for a total of 10 cycles. The solution was then extruded 10 times through a 0.22 μm polycarbonate membrane. Finally, the LUVs solution containing the HPTS probe was obtained by purification using a Sephadex G-50 dextran column with HEPES buffer solution free of the HPTS probe as an eluent. The vesicles were stored at 4° C. and used within two days to ensure uniform vesicle size. The results are shown in FIGS. 11A-11F.

As shown in FIG. 11A: The external buffer was 10 mM HEPES, pH=6.4. Influenced by the pH gradient, external protons transported from the outside to the inside of the vesicles, and the proton transport efficiency could be monitored by changes in the fluorescence intensity of the internal HPTS probe. As shown in FIG. 11B: At the same concentration (3.18 μM), H3 exhibited higher proton transport activity, reaching about 80% of that of gA within 300 s. The transport activity of H2 was approximately equal to that of H1, indicating that the polymer type had little effect on transport activity, while an increase in the degree of branching could enhance transport activity. Finally, the Hill equation Y=1/(1+(EC₅₀/[C])ⁿ) was used to determine the EC₅₀ values. It can be seen from FIG. 11C and FIG. 11D that the EC₅₀ values for gA, H3, H2, and H1 were determined to be 0.07 μM, 1.43 μM, 2.36 μM, and 2.60 μM, respectively. This indicated that the proton transport activity of H3 was particularly high, comparable to natural gramicidin (≈1/20). Almost no artificial proton channels reported previously achieved such high activity.

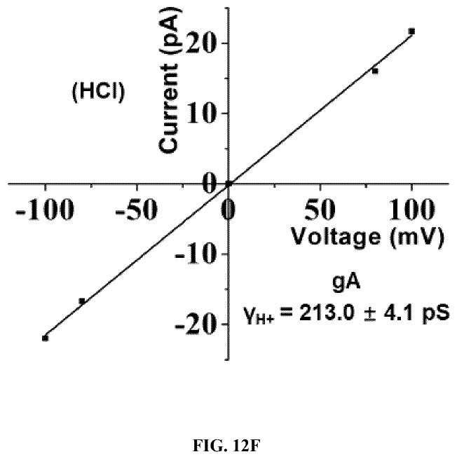

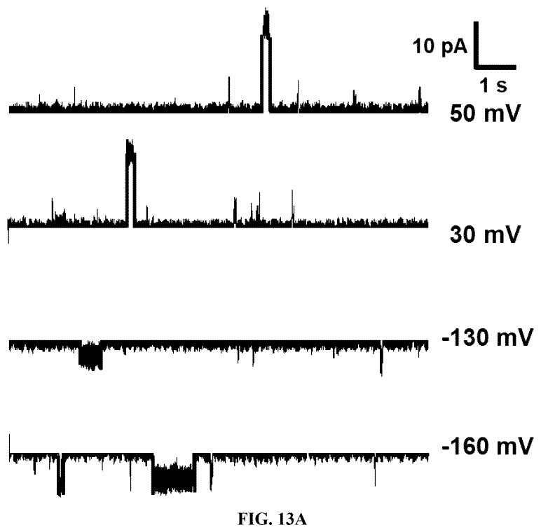

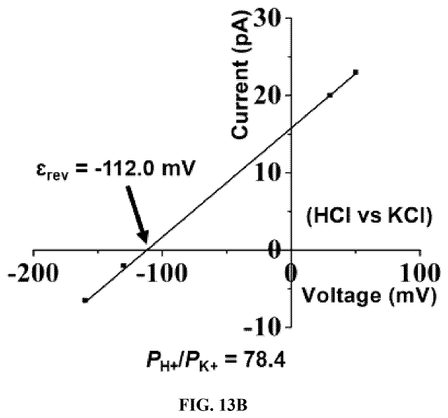

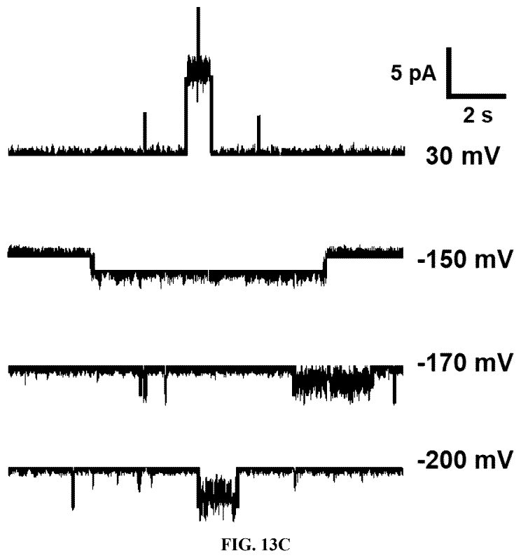

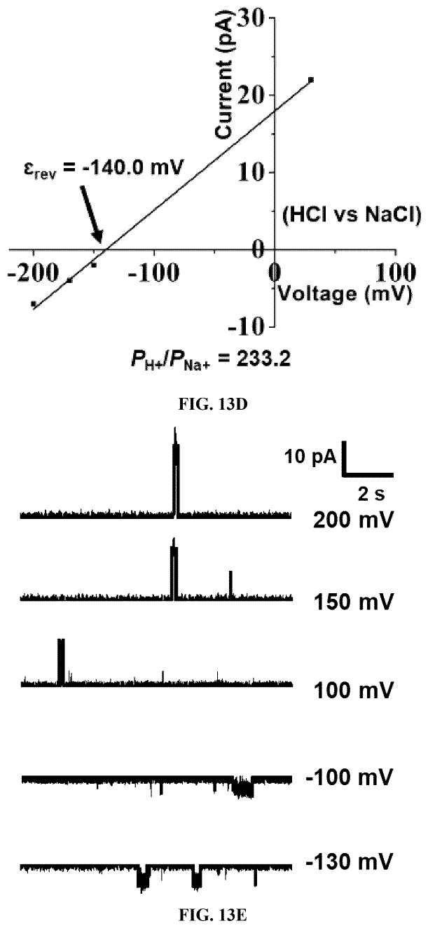

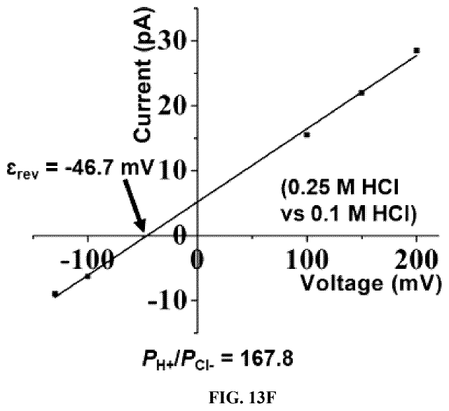

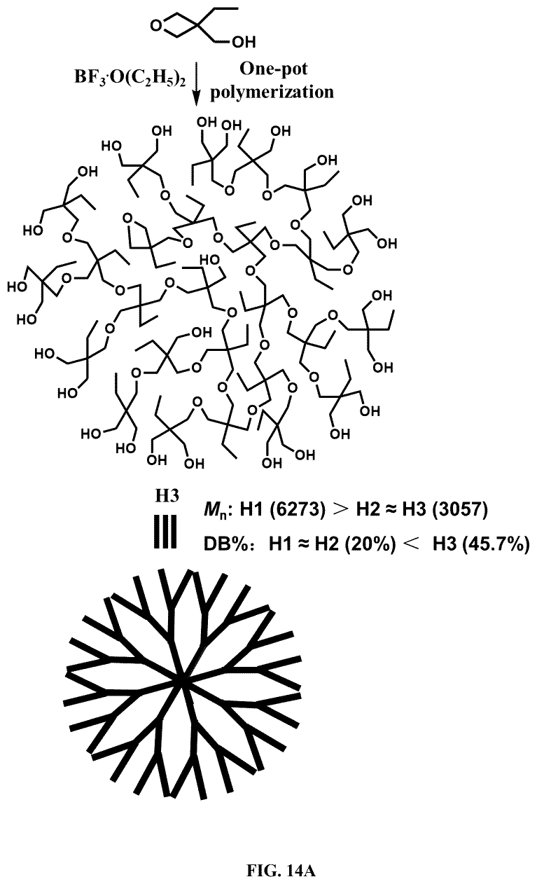

3. Lipid Bilayer Transport Mechanism, Transport Rate, and Ion Selectivity Tests: To determine whether the proton transport system functioned via a channel or carrier mechanism, planar lipid bilayer experiments were conducted. The phospholipids for the lipid bilayer were prepared as follows: First, glycerol monooleate (GMO, 15 mg) and cholesterol (15 mg) were weighed and dissolved in 20 mL of chloroform, mixed thoroughly, and aliquoted equally into 10 glass vials. The solvent was then removed under vacuum using an oil pump for 4 h, also leaving a visible film. These aliquoted lipids in the vials were stored at −20° C. Before each use, one vial was brought to room temperature, and the lipid film was dissolved in 50 μL of n-decane for use. Any unused solution was also stored at −20° C. and used within 7 d. Before testing, a membrane was painted using 0.5 μL of the prepared solution, and the test worked best when the capacitance was around 80 pF. To measure the transport rate, 0.25 M aqueous hydrochloric acid (HCl) solution was added to both the cis and trans chambers. The results are shown in FIGS. 12A-12F and FIGS. 13A-13F.

It can be seen from FIG. 12A that distinct square proton channel signals were detected, indicating that H3 transported protons via a channel mechanism rather than a carrier mechanism. From FIG. 12B, FIG. 12C, FIG. 12E, and FIG. 12F, by applying different voltages and measuring the corresponding current values, the proton conductance (γ_{H}_{⁺}) of H3 was determined to be 180.5±4.4 pS. The conductance of gA was 213.0±4.1 pS, indicating that the proton transport of H3 was very fast, on the same order of magnitude as gA. From FIG. 12D, exceptionally long and stable channel current signals were occasionally observed in this experiment, indicating that H3 could transport protons stably. The H3 channel remained continuously open for over 70 s, making it one of the most stable synthetic channels known to date.

It can be seen from FIG. 13A and FIG. 13B that finally, under asymmetric conditions with the cis chamber containing 0.25 M aqueous HCl and the trans chamber containing 0.25 M aqueous KCl, the H⁺/K⁺ selectivity (PH⁺/PK⁺) of H3 was determined using the simplified Goldman-Hodgkin-Katz (GHK) equation. Based on the recorded I-V curve and the obtained reversal potential (εrev=−112.0 mV), PH⁺/PK⁺ was calculated to be 78.4, indicating high H⁺/K⁺ selectivity for H3. From FIG. 13C and FIG. 13D, the H⁺ selectivity over Na⁺ (PH⁺/PNa⁺) was investigated by replacing the 0.25 M KCl in the trans chamber with 0.25 M NaCl. Based on the I-V curve obtained by the same method and the reversal potential (εrev=−140.0 mV), PH⁺/PNa⁺ was calculated to be 233.2. From FIG. 13E and FIG. 13F, finally, the H⁺/Cl⁻ selectivity (PH⁺/PCl⁻) was measured under asymmetric HCl conditions (cis chamber=0.25 M HCl, trans chamber=0.1 M HCl). Considering the proton gradient from 0.1 M to 0.25 M and the reversal potential derived from the I-V curve (εrev=−46.7 mV), the H⁺/Cl⁻ selectivity value for H3 reached 167.8. These results strongly confirmed that H3 not only transported protons rapidly but also exhibited significant H⁺/K⁺, H⁺/Na⁺, and H⁺/Cl⁻ selectivity.

Example 4 This example provided a selenium-containing HBP. A preparation method included the following steps:

S1. Preparation of a compound M1: A1, 0.75 g (4 mmol) of dimethyl diselenide was dissolved in 30 mL of anhydrous ethanol and stirred uniformly. 454 mg (12 mmol) of sodium borohydride was slowly added at 0° C., and the mixture was stirred at 0° C. for 30 min. The solution color changed from yellow to colorless, yielding a mixture A.

A2, 1.23 g (10 mmol) of ethyl chloroacetate was dissolved in 5 mL of anhydrous ethanol and stirred uniformly. This solution was added to the mixture A obtained in the step A1, and a resulting mixture was stirred at 0° C. for 1 h, yielding a mixture B.

A3, 50 mL of distilled water and 50 mL of anhydrous ethanol were sequentially added to the mixture B obtained in the step A2. The organic layer was separated by extraction, sequentially washed with 50 mL of distilled water and 50 mL of brine, and the organic phase was retained. The solvent was removed under reduced pressure, yielding a mixture C.

A4, The mixture C obtained in the step A3 was dissolved in 10 mL of 99 wt% ethanol, and 10 mL of an 8 mol aqueous KOH solution was added. The mixture was stirred overnight at room temperature. Then, 30 mL of distilled water and 30 mL of diethyl ether were sequentially added, followed by acidification with concentrated hydrochloric acid to pH=2. Extraction was conducted with 30 mL of diethyl ether, and the organic layer was washed with 30 mL of distilled water, dried over anhydrous Na₂SO₄, and the 99 wt % ethanol and diethyl ether was removed using a rotary evaporator to obtain the compound M1 (0.43 g, yield of 70%).

S2. Preparation of HBP: A preparation method was the same as that in Example 3, yielding an HBP H3.

S3. 224 mg (1.46 mmol) of the compound M1 obtained in the step S1 was dissolved in 10 mL of anhydrous DCM. Then, 10 μL of anhydrous DMF and 1.85 g (14.6 mmol) of oxalyl chloride were added. Under nitrogen protection, the mixture was heated under reflux at 60° C. with stirring overnight. The solvent and excess oxalyl chloride were removed under reduced pressure, yielding a reactant A.

S4. Under nitrogen protection, 20 mg (0.007 mmol) of the HBP obtained in the step S2 and 6.83 mg (0.07 mmol) of TEA were suspended in 5 mL of anhydrous THF and stirred at 0° C. for 5 min, yielding a reactant B.

S5. The reactant A obtained in the step S3 was dissolved in 2 mL of anhydrous THF and stirred uniformly. This solution was dropwise added to the reactant B obtained in the step S4. A resulting mixture was heated to room temperature, and the mixture was stirred overnight. The solvent was removed using a rotary evaporator, and a resulting product was dissolved in 1 mL of anhydrous THF. Dialysis was conducted for 24 h in a THF-water mixture system (a volume ratio of anhydrous THF to distilled water=1:1). During the dialysis, the THF-water mixture system was replaced every 3 h, and a dialysis membrane with a molecular weight cut-off of 1,000 Da was used to obtain the selenium-containing HBP H3-Se (23.2 mg, yield of 50%).

The compound M1 and the selenium-containing HBP H3-Se obtained in Example 4 were characterized by NMR and GPC.

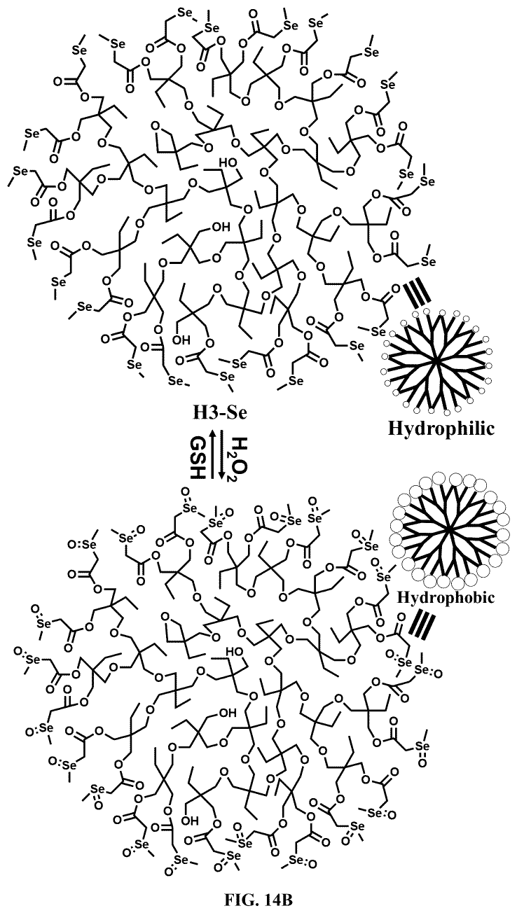

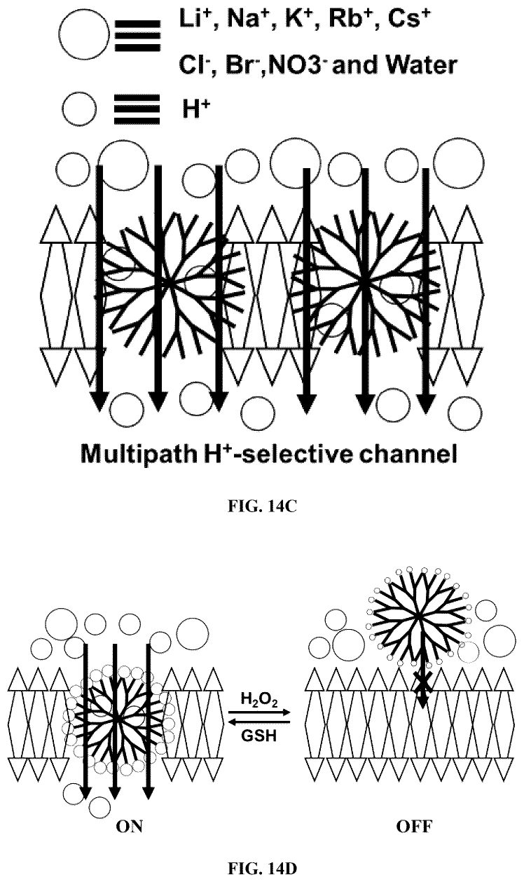

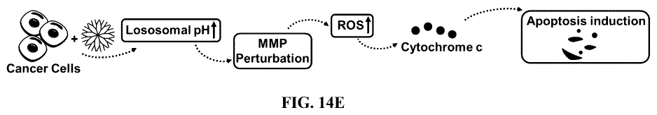

FIGS. 14A-14E show the chemical structures of H1 to H3 and H3-Se and a schematic diagram of proton transport inducing cancer cell apoptosis.

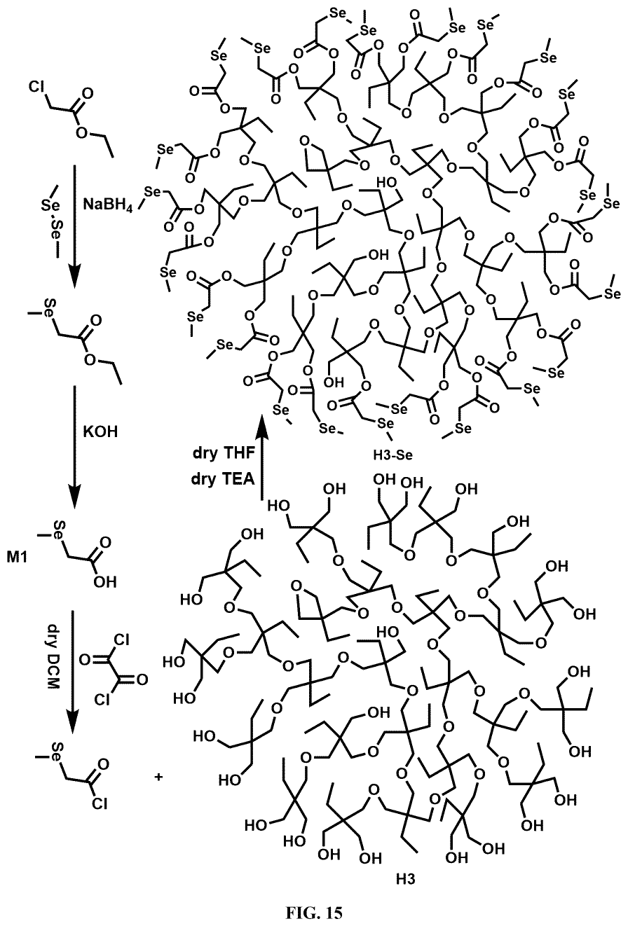

FIG. 15 shows the synthesis routes of M1 and H3-Se in Example 4.

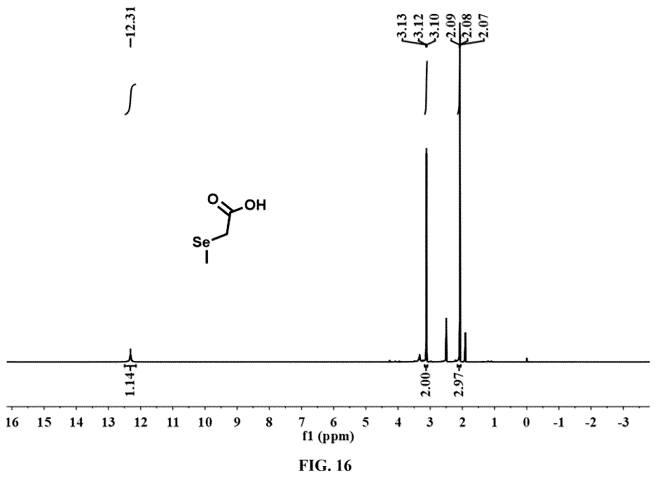

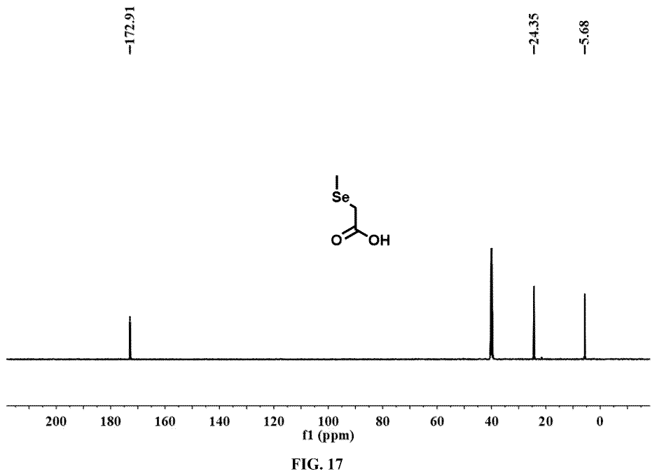

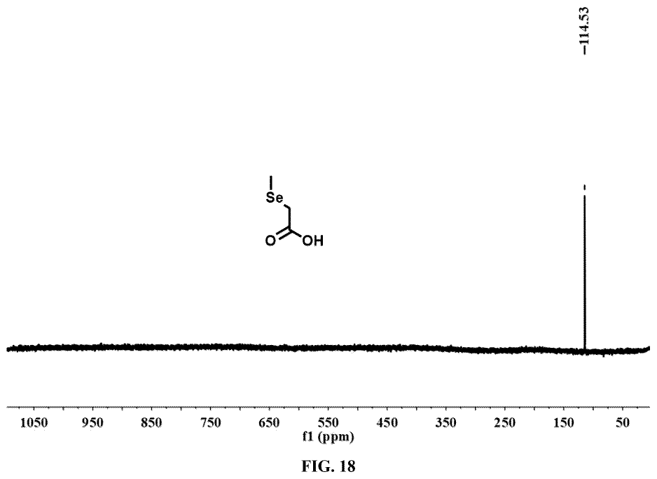

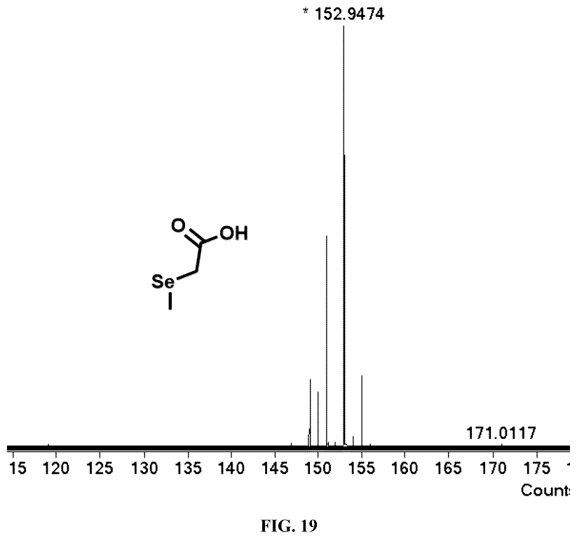

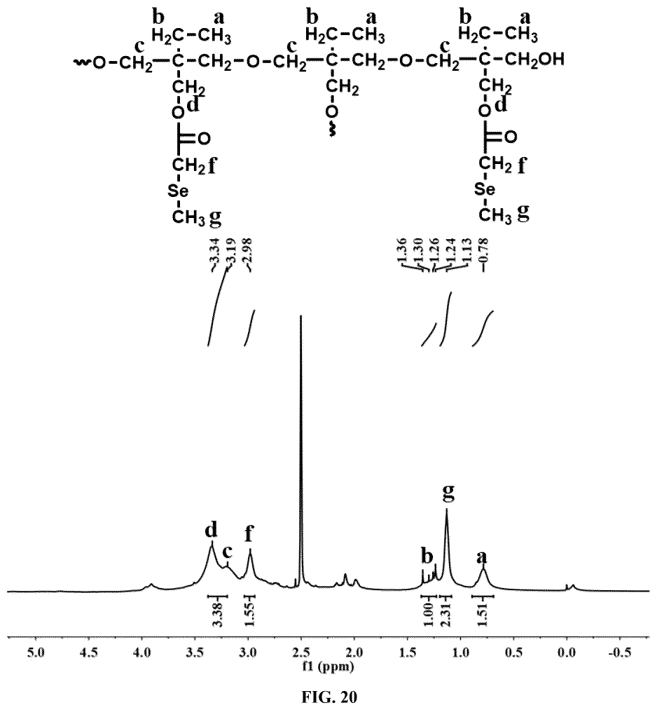

The results for M1 are shown in FIG. 16 to FIG. 19.

From FIG. 16 to FIG. 19, it was determined that M1 was successfully synthesized.

M1: ¹H NMR (500 MHz, DMSO-d6) δ 12.31 (s, 1H), 3.17-3.08 (m, 2H), 2.15-2.05 (m, 3H).

¹³C NMR (500 MHz, DMSO-d6) δ 172.91, 24.35, 5.68.

⁷⁷Se NMR (500 MHz, DMSO-d6) δ 114.53.

HR-MS (ESI, m/z): calculated for C3H6O2Se [M-H]-: 152.9449; found, 152.9449.

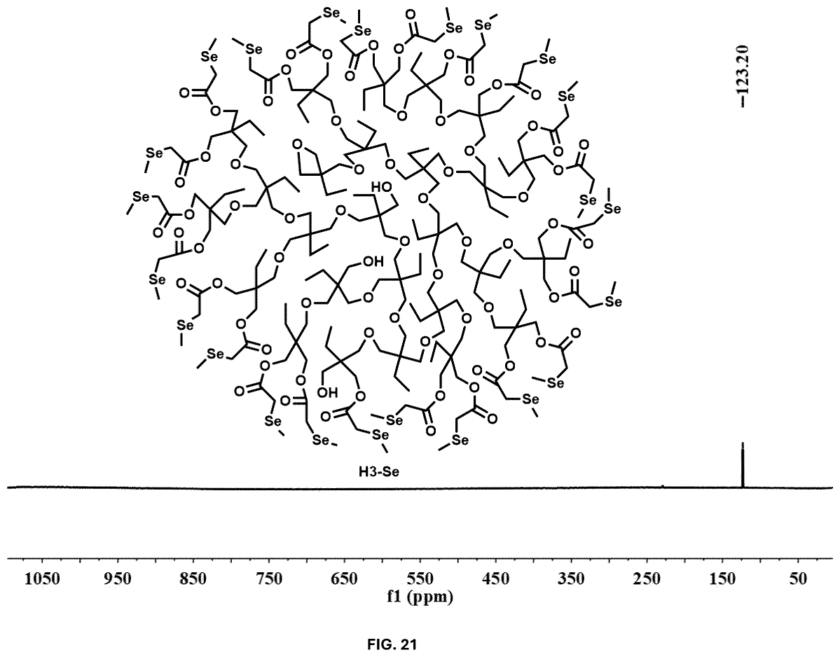

For the selenium-containing HBP H3-Se, the NMR characterization data are shown in FIG. 20 and FIG. 21.

From FIG. 20 and FIG. 21,

H3-Se: ¹H NMR (500 MHz, DMSO-d₆) δ 3.34 (s, 3H), 2.98 (s, 2H), 1.29 (dd, J=39.8, 20.2 Hz, 1H), 1.13 (s, 2H), 0.78 (s, 2H).

⁷⁷Se NMR (500 MHz, DMSO-d₆) δ 123.20.

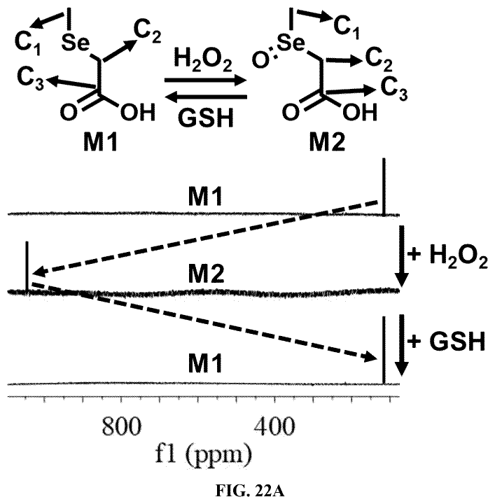

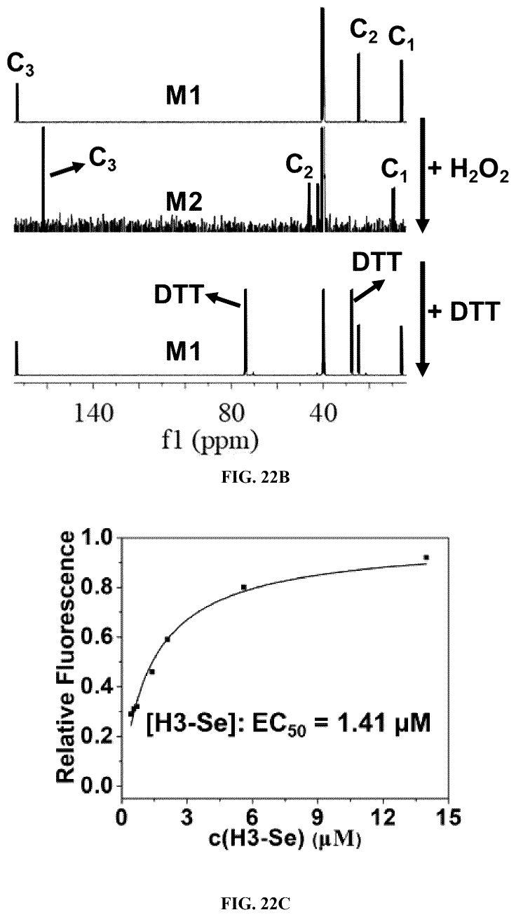

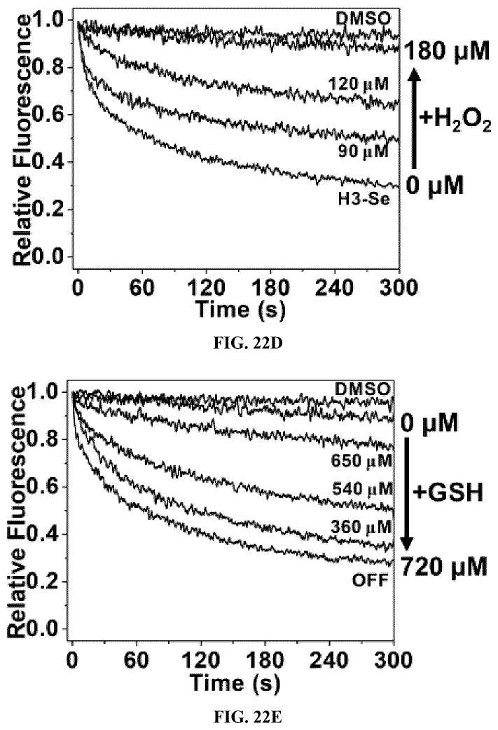

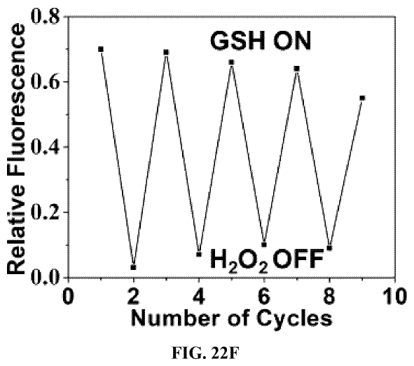

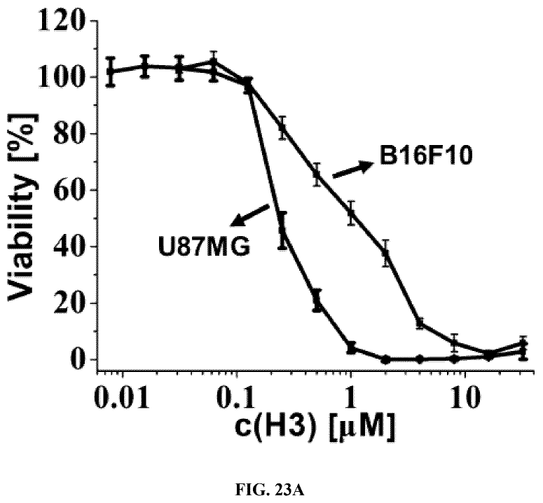

The redox behavior of compound 1 (compound M1) was analyzed by NMR spectroscopy. Subsequently, the transport activity and “ON-OFF” switching characteristics of H3-Se were evaluated using LUVs-based HPTS assays (intravesicular solution: 10 mM HEPES, pH=7.0; extravesicular solution: 10 mM HEPES, pH=7.0). The results are shown in FIGS. 22A-22F.

It can be seen from FIG. 22A that the ⁷⁷Se NMR spectrum showed a chemical shift of 114.53 ppm for M1 containing the selenoether. After oxidation of M1 with H₂O₂, the chemical shift moved to 1047.12 ppm, indicating the conversion of the selenoether to a selenoxide group. After the addition of GSH, the chemical shift returned to 114.53 ppm. From FIG. 22B, the ¹³C NMR spectrum showed significant chemical shifts for three distinct peaks of M1 (C1, C2, and C3) after the addition of H₂O₂; for example, the chemical shift of C3 changed from 172.95 ppm to 161.40 ppm. After the addition of 1,4-Dithiothreitol (DTT), the chemical shifts of these three peaks returned to their original positions, indicating a reversible transformation between the selenoether and the selenoxide.

It can be seen from FIG. 22C that the EC₅₀ value of H3-Se was 1.41 μM, indicating activity comparable to H3. To measure the redox-regulated proton transport activity of H3-Se, H3-Se was pre-incubated with different concentrations of H₂O₂ for 1 min before being added to the LUVs solution. It can be seen from FIGS. 22D-22E that the relative fluorescence intensity of the HPTS probe increased with the increasing concentration of added H₂O₂, meaning the proton transport activity decreased correspondingly. At an H₂O₂ concentration of 180 μM, the proton transport activity of H3-Se was very low, comparable to that of the DMSO control group. Subsequently, different concentrations of GSH were added to the H₂O₂-treated H3-Se solution (pre-incubated for 1 min), and the proton transport ability was restored. It can be seen from FIG. 22F that the proton transport activity of H3-Se could be cycled multiple times. It was inferred that the hydrophobic selenoether moiety of H3-Se allowed integration into the hydrophobic phospholipid bilayer, while conversion to the hydrophilic selenoxide moiety impeded this process.

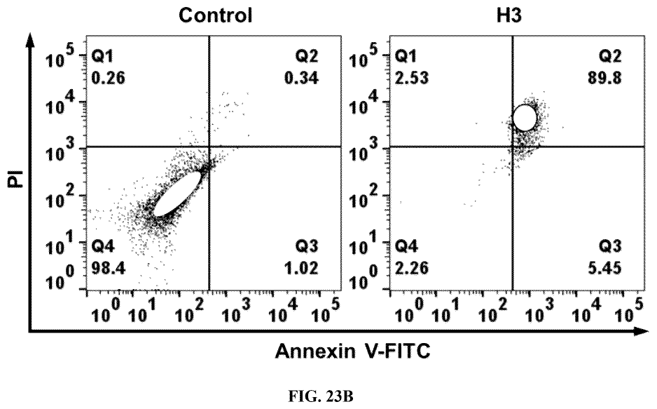

After confirming the rapid proton transport capability of H3 in the liposome model, its potential anticancer properties were investigated. First, the cytotoxicity of H3 against two cancer cell lines, melanoma B16F10 and malignant glioma U87MG, was evaluated using the Cell Counting Kit-8 (CCK-8) assay. The results are shown in FIGS. 23A-23B.

It can be seen from FIG. 23A that the half maximal inhibitory concentration (IC₅₀) of H3 for B16F10 cells was 1.04 μM, and the IC₅₀ of H3 for U87MG cells was 0.23 μM, indicating significant toxicity against both cancer cell lines. To determine whether H3 caused cell death via an apoptotic mechanism, flow cytometry experiments were conducted combined with Annexin V and propidium iodide (PI) staining. It can be seen from FIG. 23B that after 24 h of culture with H3, the percentages of early and late apoptotic cells were 5.45% and 89.8%, respectively, which were substantially higher than those in the DMSO-treated control group (1.02% and 0.34%). These experiments clearly demonstrated that H3 efficiently transported protons, altered intracellular pH, and ultimately induced cancer cell apoptosis.

Finally, it should be noted that the foregoing embodiments are only intended to describe, rather than to limit the technical solutions of the present disclosure. Although the present disclosure is described in detail with reference to the preferred embodiments, a person of ordinary skill in the art should understand that modifications or equivalent replacements may be made to the technical solutions of the present disclosure without departing from the spirit and scope of the technical solutions of the present disclosure.

What is claimed is:

1. A preparation method of a selenium-containing hyperbranched polymer (HBP), comprising following steps:
S1, preparing a compound M1;
S2, preparing an HBP;
S3, dissolving the compound M1 obtained in the step S1 in anhydrous dichloromethane (DCM), adding anhydrous N,N-dimethylformamide (DMF) and oxalyl chloride, heating and stirring an obtained mixture overnight, and removing the anhydrous DCM, the anhydrous DMF, and excess oxalyl chloride under reduced pressure to obtain a reactant A;
S4, under nitrogen protection, suspending the HBP obtained in the step S2 and triethylamine (TEA) in anhydrous tetrahydrofuran (THF), and stirring at 0° C. for 5 min to obtain a reactant B; and
S5, dissolving the reactant A obtained in the step S3 in anhydrous THF, stirring uniformly, dropwise adding an obtained solution to the reactant B obtained in the step S4, heating an obtained mixture to room temperature, stirring overnight, removing the anhydrous THF using a rotary evaporator, redissolving an obtained product in anhydrous THF, and conducting dialysis for 24 h in a THF-water mixture system with a volume ratio of 1:1 to obtain the selenium-containing HBP.

2. The preparation method according to claim 1, wherein a preparation process of the compound M1 in the step S1 comprises following steps:
A1, dissolving dimethyl diselenide in anhydrous ethanol, stirring uniformly, slowly adding sodium borohydride at 0° C., and stirring at 0° C. for 30 min to obtain a mixture A;
A2, dissolving ethyl chloroacetate in anhydrous ethanol, stirring uniformly, adding an obtained solution to the mixture A obtained in the step A1, and stirring at 0° C. for 1 h to obtain a mixture B;
A3, adding distilled water and anhydrous ethanol sequentially to the mixture B obtained in the step A2, conducting extraction to separate an organic layer, washing the organic layer with distilled water and brine sequentially, retaining an obtained organic phase, and removing the anhydrous ethanol under reduced pressure to obtain a mixture C; and
A4, dissolving the mixture C obtained in the step A3 in ethanol with a mass fraction of 99%, adding an aqueous KOH solution, stirring overnight at room temperature, adding distilled water and diethyl ether sequentially, acidifying with concentrated hydrochloric acid to a pH value of 2, conducting extraction with diethyl ether, washing an obtained organic layer with distilled water, drying the organic layer over anhydrous Na₂SO₄, and removing the ethanol with a mass fraction of 99% and the diethyl ether using a rotary evaporator to obtain the compound M1.

3. The preparation method according to claim 1, wherein a preparation process of the HBP in the step S2 comprises following steps:
B1, under nitrogen protection, adding anhydrous DCM and boron trifluoride diethyl etherate to a round-bottom flask to obtain a mixture D; and
B2, adding 3-ethyl-3-(hydroxymethyl)oxetane to the mixture D obtained in the step B1 via a dropping funnel within 5 min, conducting a reaction at −20° C. to 30° C. for 48 h to 54 h, quenching the reaction with ethanol, adding an obtained reaction product to ultrapure water, filtering, and vacuum drying at 80° C. for 180 min to obtain the HBP.

4. The preparation method according to claim 1, wherein in the step S3, the compound M1 obtained in the step S1 is dissolved in the anhydrous DCM, the anhydrous DMF and the oxalyl chloride are added, and the mixture is heated and stirred under reflux at 60° C. overnight under nitrogen protection.

5. The preparation method according to claim 1, wherein the anhydrous THF and distilled water are at a volume ratio of 1:1 in the THF-water mixture system in the step S5.

6. The preparation method according to claim 1, wherein during the dialysis in the step S5, the THF-water mixture system is replaced every 3 h, and a dialysis membrane used has a molecular weight cut-off of 1,000 Da.

7. A selenium-containing HBP prepared by the preparation method according to claim 1.

8. A pharmaceutical composition, comprising: a selenium-containing HBP prepared by the preparation method according to claim 1.

9. The pharmaceutical composition according to claim 8, comprising: the selenium-containing HBP as an active ingredient and a pharmaceutically acceptable carrier.

10. A selenium-containing HBP prepared by the preparation method according to claim 2.

11. A selenium-containing HBP prepared by the preparation method according to claim 3.

12. A selenium-containing HBP prepared by the preparation method according to claim 4.

13. A selenium-containing HBP prepared by the preparation method according to claim 5.

14. A selenium-containing HBP prepared by the preparation method according to claim 6.

15. A pharmaceutical composition, comprising: a selenium-containing HBP prepared by the preparation method according to claim 2.

16. A pharmaceutical composition, comprising: a selenium-containing HBP prepared by the preparation method according to claim 3.

17. A pharmaceutical composition, comprising: a selenium-containing HBP prepared by the preparation method according to claim 4.

18. A pharmaceutical composition, comprising: a selenium-containing HBP prepared by the preparation method according to claim 5.

19. A pharmaceutical composition, comprising: a selenium-containing HBP prepared by the preparation method according to claim 6.

20. A pharmaceutical composition, comprising: the selenium-containing HBP according to claim 7.
